(19) United States
(12) Patent Application Publication
(10) Pub. No.: US 2024/0253424 A1
(43) Pub. Date: Aug. 1, 2024

(54) VEHICLE FRAGRANCE SUPPLY APPARATUS

(71) Applicant: TOYOTA JIDOSHA KABUSHIKI KAISHA, Toyota-shi (JP)

(72) Inventor: Ryuji OTSUKA, Toyota-shi (JP)

(21) Appl. No.: 18/530,214

(22) Filed: Dec. 6, 2023

(30) Foreign Application Priority Data
Jan. 31, 2023 (JP) 2023-013598

(51) Int. Cl. B60H 3/00

(52) U.S. Cl. CPC B60H 3/0035; B60H 2003/0042

(57) ABSTRACT

A vehicle fragrance supply device, includes: a plurality of fragrance generators; a supply pipe configured to supply a fragrance generated from the plurality of fragrance generators to a vehicle cabin interior; a switching valve configured to link the plurality of fragrance generators with the supply pipe; and an external air introduction pipe connected to the switching valve and configured to introduce external air. The switching valve is switchable between a first communication state in which one fragrance generator of the plurality of fragrance generators and the supply pipe are communicated with each other, a second communication state in which at least another one fragrance generator of the plurality of fragrance generators and the supply pipe are communicated with each other, and a third communication state in which the external air introduction pipe and the supply pipe are communicated with each other.

### CROSS-REFERENCE TO RELATED APPLICATION

This application claims priority under 35 USC 119 from Japanese Patent Application No. 2023-13598, filed Jan. 31, 2023, the disclosure of which is incorporated by reference herein in its entirety.

### BACKGROUND

### Technical Field

The present disclosure relates to a vehicle fragrance supply device.

### Related Art

Japanese Patent Application Laid-open (JP-A) No. 2019-38385 discloses a fragrance generating device including a cartridge filled with different fragrances.

In the fragrance generating device described in the foregoing publication, the perfume loaded in the cartridges is discharged into the vehicle cabin by blowing air from a blower. Moreover, it is described that vanilla-based perfumes are released during heating, and mint-based perfumes are released during cooling.

### SUMMARY

The fragrance generating device described in JP-A No. 2019-38385 employs a common supply path for different cartridges, enabling space saving to be achieved. However, there is a possibility that there will be residual aroma when the perfume is changed.

An object of the present disclosure is to provide a vehicle fragrance supply device that enables space saving to be achieved while suppressing the occurrence of residual aroma.

A vehicle fragrance supply device of a first aspect of the present disclosure includes: plural fragrance generators; a supply pipe configured to supply a fragrance generated from the plural fragrance generators to a vehicle cabin interior; a switching valve configured to link the plural fragrance generators with the supply pipe; and an external air introduction pipe connected to the switching valve and configured to introduce external air, in which the switching valve is switchable among a first communication state in which one fragrance generator of the plural fragrance generators and the supply pipe are communicated with each other, a second communication state in which at least one another fragrance generator of the plural fragrance generators and the supply pipe are communicated with each other, and a third communication state in which the external air introduction pipe and the supply pipe are communicated with each other.

In the vehicle fragrance supply device of the first aspect, plural fragrance generators are provided, and the respective fragrance generators are configured so as to be capable of supplying fragrance into the vehicle cabin via the supply pipe. The fragrance generator and the supply pipe are connected by a switching valve. The switching valve is configured so as to be switchable among a first communication state in which one fragrance generator of the plural fragrance generators and the supply pipe are communicated with each other, and a second communication state in which at least one another fragrance generator of the plural fragrance generators and the supply pipe are communicated with each other. As a result, since the fragrance is supplied from the plural fragrance generators to the vehicle cabin through a common supply pipe, compared to a structure in which different supply pipes are provided for each fragrance generator, space saving can be achieved.

Further, the switching valve is connected to an external air introduction pipe through which external air is introduced, and the switching valve is configured so as to be switchable to a third communication state in which the external air introduction pipe and the supply pipe are communicated with each other. As a result, when the external air flows through the supply pipe in the third communication state, fragrance retained in the supply pipe is discharged from the supply pipe, and the occurrence of residual aroma can be suppressed.

A vehicle fragrance supply device of a second aspect of the present disclosure is the first aspect, in which the switching valve comprises a rotary valve element, by rotation of which, switching among the first communication state, the second communication state, and the third communication state is performed.

In the vehicle fragrance supply device of the second aspect, since the first communication state, the second communication state, and the third communication state can be switched by rotating the valve element, the communication state can be switched with a simple structure.

A vehicle fragrance supply device of a third aspect of the present disclosure is the first aspect, in which, in the third communication state, all of the plurality of fragrance generators are in a non-communicated state with the supply pipe.

In the vehicle fragrance supply device of the third aspect, since only the external air introduced via the external air introduction pipe flows through the supply pipe in the third communication state, residual aroma in the supply pipe can be effectively removed.

A vehicle fragrance supply device of a third aspect of the present disclosure is the first aspect, in which an air blowing device is provided at the supply pipe.

In the vehicle fragrance supply device of the fourth aspect, since the fragrance generators and the external air introduction pipe can share the air blowing device, the need to provide a dedicated blower device for introducing air from the exterior is eliminated.

A vehicle fragrance supply device of a fifth aspect of the present disclosure is any of the first to fourth aspects, in which, when transitioning between the first communication state in which one of the fragrance generators is communicated with the supply pipe and the second communication state in which another of the fragrance generators is communicated with the supply pipe, the switching valve passes through the third communication state.

In the vehicle fragrance supply device of the fifth aspect, when the fragrance is changed, external air is introduced into the supply pipe via the third communication state. As a result, since the fragrance from another fragrance generator flows into the supply pipe after any residual aroma has been removed, mixing of fragrances can be suppressed.

As described above, the vehicle fragrance supply device according to the present disclosure enables space saving to be achieved while suppressing the occurrence of residual aroma.

### BRIEF DESCRIPTION OF THE DRAWINGS

FIG. 1 is a block diagram illustrating a hardware configuration of a vehicle fragrance supply device according to an exemplary embodiment.

FIG. 2 is a schematic diagram of a vehicle fragrance supply device according to an exemplary embodiment, illustrating a state in which a first fragrance generator and a supply pipe are communicated with each other.

FIG. 3 is a schematic diagram of a vehicle fragrance supply device according to an exemplary embodiment, illustrating a state in which a second fragrance generator and a supply pipe are communicated with each other.

FIG. 4 is a schematic diagram of a vehicle fragrance supply device according to an exemplary embodiment, illustrating a state in which an external air introduction pipe and a supply pipe are in communication with each other.

### DETAILED DESCRIPTION

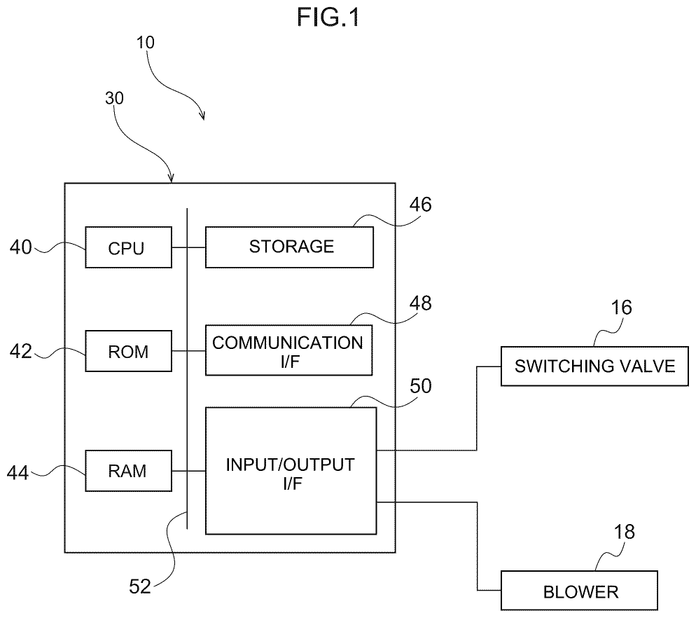

Explanation follows regarding a vehicle fragrance supply device 10 (hereafter simply referred to as the “fragrance supply device 10”) according to an exemplary embodiment, with reference to the drawings. FIG. 1 is a block diagram illustrating a hardware configuration of a fragrance supply device 10 according to an exemplary embodiment. The fragrance supply device 10 is installed in a vehicle and, for example, is disposed inside an instrument panel provided at the front of the vehicle cabin, being a device that supplies a fragrance into a vehicle cabin. As illustrated in FIG. 1, the fragrance supply device 10 of the present exemplary embodiment includes a control unit 30. The control unit 30 may be disposed in the vicinity of a fragrance supply unit that supplies the fragrance, or may be disposed at a position removed from the fragrance supply unit. The control unit 30 may be an electronic control unit (ECU) that controls the vehicle.

The control unit 30 of the fragrance supply device 10 is configured including a central processing unit (CPU: processor) 40, read only memory (ROM) 42, random access memory (RAM) 44, storage 46, a communication interface (communication I/F) 48, and an input/output interface (input/output I/F) 50. These configurations are connected together via an internal bus 52 so as to be capable of communicating with each other.

The CPU 40 is a central processing unit that executes various programs and controls various units. Namely, the CPU 40 reads a program from the ROM 42 or the storage 46, and executes the program using the RAM 44 as a workspace. The CPU 40 controls the respective configurations and performs various computation processing according to a program recorded in the ROM 42 or the storage 46.

The ROM 42 stores various programs and various data. The RAM 44 serves as a workspace to temporarily store programs and data. The storage 46 is configured by a hard disk drive (HDD) or a solid state drive (SSD), and is a non-transitory recording medium that stores various programs including an operating system, as well as various data. In the present exemplary embodiment, a program or the like for performing various processing is stored in the ROM 42 or the storage 46.

The communication I/F 48 is a communication interface used by the fragrance supply device 10 to communicate with other devices and the like, and for example employs a protocol such as CAN (Controller Area Network), Ethernet (registered trademark), LTE (Long Term Evolution), FDDI (Fiber Distributed Data Interface), or Wi-Fi (registered trademark).

The input/output I/F 50 is connected to a switch valve 16 and a blower 18 serving as a blower device. Details of the switch valve 16 and the blower 18 are described later.

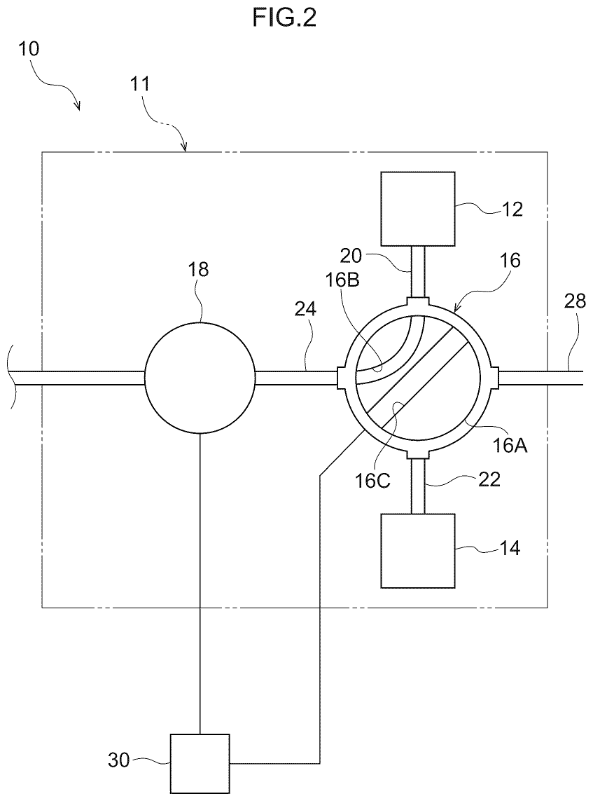

FIG. 2 is a schematic diagram of the fragrance supply device 10 according to the present exemplary embodiment. As illustrated in FIG. 2, the fragrance supply device 10 includes a fragrance supply unit 11. The fragrance supply unit 11 is mainly configured including a first fragrance generator 12, a second fragrance generator 14, a switch valve 16, a blower 18, and an external air introduction pipe 28.

Although the present exemplary embodiment includes two fragrance generators, namely, the first fragrance generator 12 and the second fragrance generator 14 as an example, there is no limitation to this, and a configuration including three or more fragrance generators may be employed. The first fragrance generator 12 and the second fragrance generator 14 are configured so as to be able to load a cartridge (not illustrated) containing an aromatic agent. This enables replenishment or replacement of the aromatic agent merely by replacing the cartridge.

One end of a first connecting pipe 20 is connected to the first fragrance generator 12, and the other end of the first connecting pipe 20 is connected to the switch valve 16. Namely, the first connecting pipe 20 connects the first fragrance generator 12 and the switch valve 16 together.

The second fragrance generator 14 is disposed on the opposite side from the first fragrance generator 12 with the switch valve 16 interposed therebetween. Note that there is no particular limitation on the positions of the first fragrance generator 12 and the second fragrance generator 14.

One end of a second connecting pipe 22 is connected to the second fragrance generator 14, and the other end of the second connecting pipe 22 is connected to the switch valve 16. Namely, the second connecting pipe 22 connects the second fragrance generator 14 and the switch valve 16 together.

The first connecting pipe 20, the second connecting pipe 22, the supply pipe 24, and the external air introduction pipe 28 are connected to the switch valve 16. The supply pipe 24 is a pipe extending from the switch valve 16 toward the vehicle cabin, and the blower 18 is provided at the supply pipe 24. The supply pipe 24 is opened in the vicinity of a non-illustrated register provided in an instrument panel. As a result, by operation of the blower 18, air inside the supply pipe 24 is supplied into the vehicle cabin. Namely, the fragrance generated from the first fragrance generator 12 and the second fragrance generator 14 is supplied into the vehicle cabin by flowing through the supply pipe 24.

The external air introduction pipe 28 is connected at one end to the switch valve 16, and is open at the other end to external air. This enables external air to be introduced via the external air introduction pipe 28. Note that the term “external air” used herein is not limited to the atmosphere outside the vehicle, and is a concept that broadly encompasses air outside the fragrance supply device 10. In the present exemplary embodiment, the other end of the external air introduction pipe 28 is open to the outside of the vehicle cabin.

The switch valve 16 is configured so as to be able to switch a state of communication between each of the first fragrance generator 12, the second fragrance generator 14, and the external air introduction pipe 28, and the supply pipe 24. More specifically, the switch valve 16 is provided with a rotary valve element 16A, and a first flow path 16B and a second flow path 16C are formed at the valve element 16A. Note that although the first flow path 16B is curved and the second flow path 16C is formed in a straight line in the present exemplary embodiment as an example, there is no limitation to this, and the first flow path 16B and the second flow path 16C may be formed in other shapes. For example, the first flow path 16B and the second flow path 16C may be formed in a substantially straight line shape, and may be respectively formed at different positions in the depth direction of the valve element 16A. Further, three or more flow paths may be formed.

The valve element 16A is electrically connected to the control unit 30, and is rotated based on a signal from the control unit 30. In the present exemplary embodiment, the valve element 16A of the switch valve 16 is rotated so as to be switchable among a first communication state or a second communication state in which one of the first fragrance generator 12 or the second fragrance generator 14, and the supply pipe 24, are communicated with each other, and a third communication state in which the external air introduction pipe 28 and the supply pipe 24 are communicated with each other. Explanation follows regarding the first communication state and the second communication state.

FIG. 2 is a diagram illustrating a first communication state in which the first fragrance generator 12 and the supply pipe 24 are communicated with each other. More specifically, one end of the first flow path 16B of the valve element 16A is connected to the first fragrance generator 12 via the first connecting pipe 20, and the other end of the first flow path 16B is connected to the supply pipe 24. Accordingly, when the control unit 30 actuates the blower 18 in the state illustrated in FIG. 2, the fragrance generated by the first fragrance generator 12 flows through the supply pipe 24 via the first flow path 16B, and is supplied into the vehicle cabin.

Moreover, in the state of FIG. 2, the second connecting pipe 22, to which the second fragrance generator 14 is connected, is closed by the valve element 16A. Accordingly, the fragrance generated by the second fragrance generator 14 is not supplied to the supply pipe 24, and the second fragrance generator 14 and the supply pipe 24 are in a non-communicated state. Moreover, the external air introduction pipe 28 is closed by the valve element 16A, not being communicated with the second flow path 16C. Accordingly, external air is not supplied to the supply pipe 24.

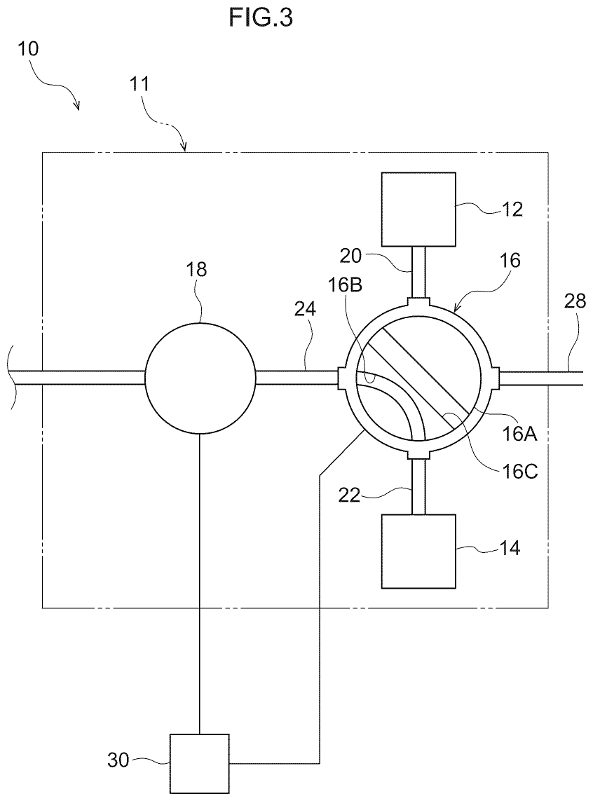

FIG. 3 is a diagram illustrating a second communication state in which the second fragrance generator 14 and the supply pipe 24 are communicated with each other. As illustrated in FIG. 3, one end of the first flow path 16B of the valve element 16A is connected to the second fragrance generator 14 via the second connecting pipe 22, and the other end of the first flow path 16B is connected to the supply pipe 24. Accordingly, when the control unit 30 actuates the blower 18 in the state illustrated in FIG. 3, the fragrance generated by the second fragrance generator 14 flows through the supply pipe 24 via the first flow path 16B, and is supplied into the vehicle cabin.

Moreover, in the state of FIG. 3, the first connecting pipe 20, to which the first fragrance generator 12 is connected, is closed by the valve element 16A. Accordingly, the fragrance generated by the first fragrance generator 12 is not supplied to the supply pipe 24, and the first fragrance generator 12 and the supply pipe 24 are in a non-communicated state. Moreover, the external air introduction pipe 28 is closed by the valve element 16A, not being communicated with the second flow path 16C. Accordingly, external air is not supplied to the supply pipe 24.

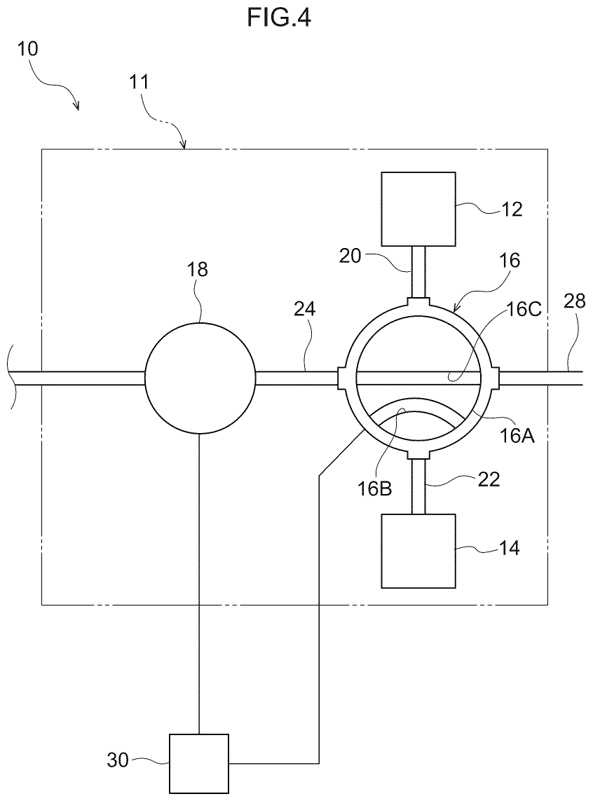

FIG. 4 is a diagram illustrating a third communication state in which the external air introduction pipe 28 and the supply pipe 24 are communicated with each other. As illustrated in FIG. 4, one end of the second flow path 16C of the valve element 16A is connected to the external air introduction pipe 28, and the other end of the second flow path 16C is connected to the supply pipe 24. Accordingly, when the control unit 30 actuates the blower 18 in the state illustrated in FIG. 4, external air introduced from the external air introduction pipe 28 flows through the supply pipe 24 via the second flow path 16C, and is supplied into the vehicle cabin.

Moreover, in the state of FIG. 4, the first flow path 16B of the valve element 16A is not connected to either the first connecting pipe 20 or the second connecting pipe 22. Accordingly, the first connecting pipe 20, to which the first fragrance generator 12 is connected, is closed by the valve element 16A, and the fragrance generated by the first fragrance generator 12 is not supplied to the supply pipe 24. Similarly, the second connecting pipe 22, to which the second fragrance generator 14 is connected, is closed by the valve element 16A, and the fragrance generated by the second fragrance generator 14 is not supplied to the supply pipe 24. As described above, in the third communication state, all of the plural fragrance generators are in a non-communicated state with the supply pipe 24.

### (Operation)

Next, explanation follows regarding the operation of the present exemplary embodiment.

According to the vehicle fragrance supply device 10 of the present exemplary embodiment, the first fragrance generator 12 and the second fragrance generator 14 are provided, and the respective fragrance generators are configured so as to be capable of supplying fragrance into the vehicle cabin through the supply pipe 24. Moreover, the first fragrance generator 12 and the second fragrance generator 14 are each connected to the supply pipe 24 by the switch valve 16. As illustrated in FIG. 2 and FIG. 3, the switch valve 16 is configured so as to be switchable to a first communication state or a second communication state in which one of the first fragrance generator 12 or the second fragrance generator 14 is communicated with the supply pipe 24. This enables the fragrance to be supplied from the first fragrance generator 12 and the second fragrance generator 14 into the vehicle cabin through the shared supply pipe 24, and compared to a structure in which the first fragrance generator 12 and the second fragrance generator 14 are provided with different supply pipes, space saving can be achieved.

As illustrated in FIG. 4, the switch valve 16 is configured so as to be switchable to a third communication state in which the external air introduction pipe 28 and the supply pipe 24 are communicated with each other. As a result, in the third communication state, the external air flows through the supply pipe 24, any scent retained in the supply pipe 24 is discharged from the supply pipe 24, and the occurrence of residual aroma can be suppressed. As a result, the fragrance supply device 10 of the present exemplary embodiment enables space saving to be achieved while suppressing the occurrence of residual aroma.

Moreover, in the present exemplary embodiment, since the first communication state, the second communication state, and the third communication state are switched by rotating the valve element 16A of the switch valve 16, the communication state can be switched with a simple structure.

Moreover, in the present exemplary embodiment, since the first connecting pipe 20 and the second connecting pipe 22 are closed by the valve element 16A in the third communication state, only the external air introduced via the external air introduction pipe 28 flows through the supply pipe 24. This enables residual aroma in the supply pipe 24 to be effectively removed.

Moreover, since the blower 18 is provided at the supply pipe 24 in the present exemplary embodiment, and since the first fragrance generator 12, the second fragrance generator 14, and the external air introduction pipe 28 can share the blower 18, the need to provide a dedicated blower device for introducing air from the exterior is eliminated.

Although explanation has been given regarding the vehicle fragrance supply device according to the exemplary embodiment, needless to say, various embodiments may be implemented within a range not departing from the scope of the present disclosure. For example, in the exemplary embodiment described above, in a case in which an instruction from an occupant to release a different fragrance has been received, the first communication state illustrated in FIG. 2 is switched to the second communication state illustrated in FIG. 3; however, at this time, the third communication state may be passed through. In the first communication state illustrated in FIG. 2, since the supply pipe 24 is filled with the fragrance generated by the first fragrance generator 12, by passing through the third communication state before transitioning to the second communication state in FIG. 3, external air introduced via the external air introduction pipe 28 flows into the supply pipe 24. As a result, when the second communication state illustrated in FIG. 3 has been switched to, the fragrance of the first fragrance generator 12 does not remain in the supply pipe 24, and therefore, mixing of fragrances can be suppressed.

Although the switch valve 16 of the above exemplary embodiment is configured using the valve element 16A including the first flow path 16B and the second flow path 16C, there is no limitation thereto. For example, an electromagnetic valve including plural ports may be used. However, it is preferable to use a rotary valve element in view of easy control.

Moreover, although explanation has been given regarding a configuration including the blower 18 as a blower device in the above exemplary embodiment, there is no limitation to this, and a configuration not including the blower 18 may be adopted. In this case, the fragrance may be supplied into the vehicle cabin using a blowing mechanism external to the fragrance supply device 10.

What is claimed is:

1. A vehicle fragrance supply device, comprising:
a plurality of fragrance generators;
a supply pipe configured to supply a fragrance generated from the plurality of fragrance generators to a vehicle cabin interior;
a switching valve configured to link the plurality of fragrance generators with the supply pipe; and
an external air introduction pipe connected to the switching valve and configured to introduce external air,
wherein the switching valve is switchable between a first communication state in which one fragrance generator of the plurality of fragrance generators and the supply pipe are communicated with each other, a second communication state in which at least another one fragrance generator of the plurality of fragrance generators and the supply pipe are communicated with each other, and a third communication state in which the external air introduction pipe and the supply pipe are communicated with each other.

2. The vehicle fragrance supply device of claim 1, wherein the switching valve comprises a rotary valve element, by rotation of which, switching among the first communication state, the second communication state, and the third communication state is performed.

3. The vehicle fragrance supply device of claim 1, wherein, in the third communication state, all of the plurality of fragrance generators are in a non-communicated state with the supply pipe.

4. The vehicle fragrance supply device of claim 1, wherein an air blowing device is provided at the supply pipe.

5. The vehicle fragrance supply device of claim 1, wherein, when transitioning between the first communication state in which one of the fragrance generators is communicated with the supply pipe and the second communication state in which another one of the fragrance generators is communicated with the supply pipe, the switching valve passes through the third communication state.
